(19) United States
(12) Patent Application Publication
(10) Pub. No.: US 2008/0181813 A1
(43) Pub. Date: Jul. 31, 2008

(54) Novel Mercaptan-Based Corrosion Inhibitors

(75) Inventors: Michael D. Greaves, Houston, TX (US); Carlos M. Menendez, Houston, TX (US); Qingjiang Meng, Sugar Land, TX (US)

Correspondence Address: MADAN, MOSSMAN & SRIRAM, P.C., 2603 AUGUSTA DRIVE, SUITE 700, HOUSTON, TX, 77057-5662, US

(73) Assignee: BAKER HUGHES INCORPORATED, Houston, TX (US)

(21) Appl. No.: 11/869,087

(22) Filed: Oct. 9, 2007

Related U.S. Application Data
(60) Provisional application No. 60/886,819, filed on Jan. 26, 2007.

(51) Int. Cl. C23F 11/00; C07C 321/04

(52) U.S. Cl. 422/7; 568/66

(57) ABSTRACT

Corrosion of both ferrous and non-ferrous metals, induced by a variety of corrosive aqueous-based environments, may be inhibited or controlled through use of corrosion inhibiting compositions comprising at least one member of certain formula-specified categories of mercaptan-based compounds. Where the compounds are appropriately selected, and particularly at low inhibitor concentrations, the compositions may inhibit corrosion to a degree that is comparable to or significantly greater than the inhibition provided by an equal amount of certain other corrosion inhibitor compounds. It is emphasized that this abstract is provided to comply with the rules requiring an abstract which will allow a searcher or other reader to quickly ascertain the subject matter of the technical disclosure. It is submitted with the understanding that it will not be used to interpret or limit the scope or meaning of the claims.

### CROSS-REFERENCE TO RELATED APPLICATIONS

This application claims priority from U.S. Provisional Patent Application Ser. No. 60/886,819 filed Jan. 26, 2007.

### BACKGROUND OF THE INVENTION

1. Field of the Invention

This invention relates to controlling the corrosion of metals. More particularly, this invention relates to compositions and methods for inhibiting corrosion of ferrous and non-ferrous metals, including alloys, in aqueous environments.

2. Background Art

It is widely known that both ferrous and non-ferrous metals, including alloys, are subject to corrosion under certain circumstances. Corrosion is generally defined as any deterioration of essential properties in a material due to chemical interaction with its environment, and in most situations it is considered to be undesirable. On a molecular level, corrosion is the consequence of the loss of an electron of a metal as it reacts with, in many cases, water and oxygen, and/or other oxygenating agents. The result of these interactions is usually formation of an oxide and/or a salt of the original metal. In most cases corrosion comprises the dissolution of a material. It may also be caused by exposure to corrosive chemicals, including, for example, acids, bases, dehydrating agents, halogens and halogen salts, organic halides and organic acid halides, acid anhydrides, and some organic materials such as phenol.

In order to combat corrosion, it is well known to treat, contact, or surround any susceptible metal, i.e., any having a thermodynamic profile that is relatively favorable to corrosion, with a so-called corrosion inhibitor. Because the efficacy of any particular corrosion inhibitor is generally known to be dependent upon the circumstances under which it is used, a wide variety of corrosion inhibitors have been developed and targeted for use. One target of great economic interest is the treatment of crude oils and gas systems, for protecting the variety of ferrous and non-ferrous metals needed for obtaining and processing the oils and gases. Such metals are present in oil and gas wells, including, for example, production and gathering pipelines, where the metals may be exposed to a variety of acids, acid gases such as CO₂ and H₂S, bases, and brines of various salinities. Other applications include industrial water treatments, construction materials, coatings, and the like. In some cases the corrosion inhibitors are desirably tailored for inhibiting specific types of corrosion, and/or for use under particular conditions of temperature, pressure, shear, and the like, and/or for inhibiting corrosion on a generalized or localized basis.

A number of corrosion inhibitors featuring sulfur containing compounds have been described. For example, U.S. Pat. No. 5,863,415 discloses that thiophosphorus compounds of a specific formula are particularly useful for corrosion inhibition in hot liquid hydrocarbons and may be used at concentrations that add to the fluid less of the catalyst-impairing phosphorus than some other phosphorus-based corrosion inhibitors. These thiophosphorus compounds also offer the advantage of being able to be prepared from relatively low cost starting materials.

Other sulfur-containing compounds are disclosed in, for example, U.S. Pat. No. 5,779,938, which describes corrosion inhibitors that are reaction products of one or more tertiary amines and certain carboxylic acids, preferably a mixture of mercaptocarboxylic and carboxylic acids. The use of sulphydryl acid and imidazoline salts are disclosed as inhibitors of carbon corrosion of iron and ferrous metals in WO 98/41673. Corrosion of iron is also addressed in WO 99/39025, which describes using allegedly synergistic compositions of polymethylene-polyaminodipropion-amides associated with mercaptoacids. A number of specifically sulfur-containing compounds are currently in commercial use as corrosion inhibitors for certain types of systems.

In view of the above, it would be desirable in the art to identify additional methods and compositions for inhibiting or controlling corrosion of both ferrous and non-ferrous metals and that, in particular, may be useful in treating hydrocarbon-containing aqueous systems. As used herein, ferrous metals include, in some non-limiting embodiments, iron and steel.

### BRIEF DESCRIPTION OF THE DRAWINGS

FIG. 1 is a plot comparing corrosion rate performance of an inventive corrosion inhibitor and a conventional corrosion inhibitor at comparable concentrations.

### SUMMARY OF THE INVENTION

In one aspect the present invention provides a method of inhibiting corrosion of metals, comprising contacting a metal in an aqueous environment wherein the metal is corrodible and a corrosion inhibitor composition comprising at least one compound selected from the group consisting of compounds adhering to one of the general formulas:

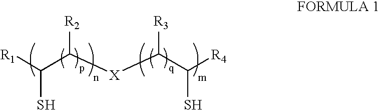

wherein x is carbon, oxygen, nitrogen, or sulfur; R₁, R₂, R₃, and R₄ are independently hydrogen or methyl, m and n are independently integers from 1 to 5, and p and q are independently integers from 1 to 4;

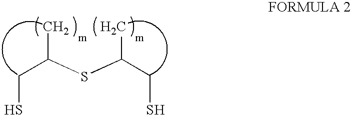

wherein m is an integer from 3 to 4; and

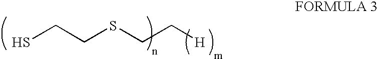

wherein m is an integer from 1 to 4 and n=4−m.

In another aspect the invention provides a method of inhibiting corrosion of metals, comprising contacting a metal in a water-containing hydrocarbon or gas stream wherein the metal is corrodible and an effective amount of a corrosion inhibitor composition comprising at least one compound selected from the group consisting of compounds adhering to one of the preceding Formulas.

In still another aspect the invention provides a composition for inhibiting corrosion of metals in an environment wherein the metal is corrodible, comprising a compound selected from the group consisting of compounds adhering to one of the preceding Formulas.

### DETAILED DESCRIPTION OF THE INVENTION

The novel mercaptan-based corrosion inhibitors identified in the present invention have been found to be efficacious in inhibiting corrosion of both ferrous and non-ferrous metals, including elemental metals, metals under conditions where passivation is inhibited, such as mild steel, stainless steel, copper and other alloys; alloys such as brasses; mixtures thereof; and the like. These mercaptan-based corrosion inhibiting compositions may be used alone, in mixtures of one or more of those defined hereinbelow, or in mixtures including other known corrosion inhibitors. They are conveniently termed “mercaptan-based”, alternatively referred to as thiols, because each category includes organosulfur molecules having at least one —SH group (a “thiol” or “sulphydryl” group), though in many embodiments these compounds contain a plurality of —SH groups.

The first group of novel mercaptan-based corrosion inhibiting compositions is defined as compounds adhering to the general formula

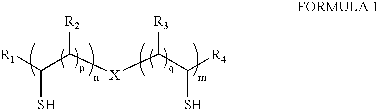

wherein x is carbon, oxygen, nitrogen, or sulfur; R₁, R₂, R₃, and R₄ are independently hydrogen or methyl; m and n are independently integers from 1 to 5; and p and q are independently integers from 1 to 4. Specific but non-limiting examples of this group include bis-2(-mercapto-1-methylpropyl)sulfide, 2-mercaptoethyl sulfide, 2-mercaptoethyl ether, 1,5-pentane dithiol, and the like.

The second group of compositions is defined as compounds adhering to the general formula

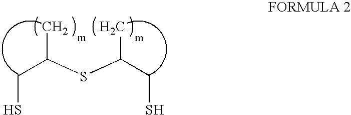

wherein m is an integer from 3 to 4. Specific but non-limiting examples of this group include bis-(2-mercaptocyclopentyl) sulfide, bis-2(2-mercaptocyclohexyl) sulfide, and the like.

The third group of compounds adheres to the general formula

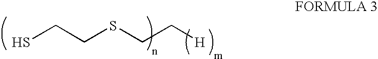

wherein m is an integer from 1 to 4 and n=4−m. Specific but non-limiting examples of this group include tetrakis-(4-mercapto-2-thiabutyl)methane and the like.

The above groups of compounds may be prepared by any means and methods known to those skilled in the mercaptan preparation art, including but not limiting to selection of sulfur-containing starting materials and sulfonation of non-sulfur-containing starting materials. Examples of non-limiting methods include those described in Buter, J. and Kellogg, R. M., “Synthesis of Sulfur-Containing Macrocycles Using Cesium Thiolates,” J. Org. Chem., 1981, 46, 4481-4485, Ochrymowycz, L. A., Mak, C-P., Michna, J. D., “Synthesis of Macrocyclic Polythiaethers,” J. Org. Chem., Vol. 39, No. 14, 1974, 2079-2084; and Gerber, D., Chongsawangvirod, P., Leung, A., Ochrymowycz, L. A., “Monocyclic Polythiaether 1,4,7-Trithiacyclononane,” J. Org. Chem., 1977, 42, 2644-2645; all of which are incorporated herein by reference in their entireties.

The corrosion inhibiting groups, and species thereof, defined hereinabove may be used for the purpose of inhibiting corrosion in any ferrous or non-ferrous metals, in both elemental and alloyed form. In certain non-limiting embodiments, examples of these metals include, but are not limited to, commonly used structure metals such as aluminum; transition metals such as iron, zinc, nickel, and copper; and combinations of these. In one non-limiting embodiment the selected material for which corrosion inhibition is desired is an alloy, such as a copper alloy or steel.

The corrosion inhibiting composition may be incorporated into the environment to which the corrodible material will be, or is being, exposed. Such environment, which includes some proportion of water, may be, in certain non-limiting embodiments, a brine, a hydrocarbon producing system such as a crude oil or a fraction thereof, or a wet hydrocarbon containing gas, such as may be obtained from an oil and/or gas well. It may be used in any proportion sufficient to accomplish the desired degree of corrosion inhibition. Such may vary from a part per billion (ppb) level to a percentage. For example, in some embodiments the corrosion inhibiting composition may vary from 100 ppb to 10,000 parts per million (ppm) in a water-containing liquid or gas hydrocarbon stream. In other embodiments the corrosion inhibiting composition may be used in an amount of from about 1 ppb to about 1 percent by volume in such hydrocarbon stream. Those skilled in the art will be able to carry out the routine experimentation needed to determine the effective level for a given corrodible material in a given environment.

The corrosion inhibiting compositions described herein may be, prior to incorporation into or with a given corrosive environment, in gas, liquid or solid form. If a solid form is used, such is desirably comminuted to a degree adequate to enable desirably controlled dissolution and/or dispersal in the corrosive environment. While particle size is not critical in the present invention, it has been found convenient to employ a corrosion inhibiting composition having particles whose diameter, in non-limiting embodiments, is from about 0.2 mm to about 1.5 mm, for employment in a corrosive environment at approximately ambient temperature. Higher temperatures will generally allow for equivalent rates of dissolution or dispersal of larger particles, while lower temperatures may necessitate smaller particles.

Incorporation of the novel corrosion inhibiting compositions of the invention may be by any means known to be effective by those skilled in the art. Simple dumping, such as into a drilling mud pit; addition via tubing in a suitable carrier fluid, such as water or an organic solvent; injection; or any other convenient means may be adaptable to these compositions. Large scale environments such as those that may be encountered in oil production, combined with a relatively turbulent environment, may not require additional measures, after or during, to ensure complete dissolution or dispersal of the corrosion inhibiting composition. In contrast, smaller, less turbulent environments, such as relatively stagnant settling tanks, may benefit from mechanical agitation of some type to optimize the performance of the corrosion inhibiting composition. Those skilled in the art will be readily able to determine appropriate means and methods in this respect.

Performance of a given corrosion inhibiting composition may be tested using any of a variety of methods, such as those specified by the American Society for Testing Materials (ASTM). One effective method, that tests the performance of a composition under conditions of moderate shear, involves a rotating coupon electrochemical technique described in ASTM: Standard Guide for Evaluating and Qualifying Oilfield and Refinery Corrosion Inhibitors in the Laboratory (Designation G170-01a), and also in NACE Publication 5A195, Item No. 24187, “State of the Art Report on Controlled-Flow Laboratory Corrosion Tests.” In this test, various concentrations of inhibitor chemistries are introduced into a given prospective, corrosive environment. The coupons are then rotated at high speed in the environment to generate moderate stress of their surfaces. Electrochemical techniques, such as, for example, linear polarization resistance, are then employed under these moderate shear conditions, to monitor the prevailing general corrosion rate as well as to identify instances of localized corrosion. A concentration profile is then generated in order to establish the minimum effective concentration of the corrosion inhibiting composition that is required to adequately protect the coupon at an acceptable corrosion rate.

The description hereinabove is intended to be general and is not intended to be inclusive of all possible embodiments of the invention. Similarly, the examples hereinbelow are provided to be illustrative only and are not intended to define or limit the invention in any way. Those skilled in the art will be fully aware that other embodiments within the scope of the claims will be apparent, from consideration of the specification and/or practice of the invention as disclosed herein. Such other embodiments may include selections of specific mercaptan-based compounds falling within the defined groups, and combinations of such compounds; proportions of such compounds; mixing and usage conditions, vessels, and protocols; hydrocarbon fluids and other fluids in which the corrosion inhibitor compositions may be used; performance in inhibiting or controlling corrosion; and the like; and those skilled in the art will recognize that such may be varied within the scope of the appended claims hereto.

### EXAMPLES

### Comparative Example 1

Two mercaptan-based compounds are compared via testing done using the procedure described in ASTM: Standard Guide for Evaluating and Qualifying Oilfield and Refinery Corrosion Inhibitors in the Laboratory (Designation G170-01a), and also in NACE Publication 5A195, Item No. 24187, “State of the Art Report on Controlled-Flow Laboratory Corrosion Tests.” One inhibitor composition contains 2-mercaptoethyl sulfide, which conforms to one embodiment of Formula 1 provided hereinabove. The other composition features an amide/imidazoline-based corrosion inhibitor and is used herein for comparative purposes.

The method includes use of a rotating cylinder electrode (RCE), a standard RCE coupon, and tube/cylinder C1018 supplied by Metal Samples, Inc. of Alabama. The corrosive environment is a combination of brine and a paraffinic hydrocarbon in an approximate weight proportion of 80:20, respectively.

During the test the coupons are rotated at approximately 6,000 revolutions per minute (rpm), and a temperature of approximately 160° F. is maintained in the corrosive environment. The test is carried out over a 17-hour time period.

The corrosion rate of the coupons is monitored electrochemically, by means of a linear polarization resistance (LPR) apparatus. To ensure that the comparison is fair, the amount of active inhibitor on a parts per million (ppm) basis in each of the inhibitor compositions is approximately the same.

The coupons are examined, post-testing, for localized corrosion using an optical microscope. Corrosion is measured as mils per year (mpy).

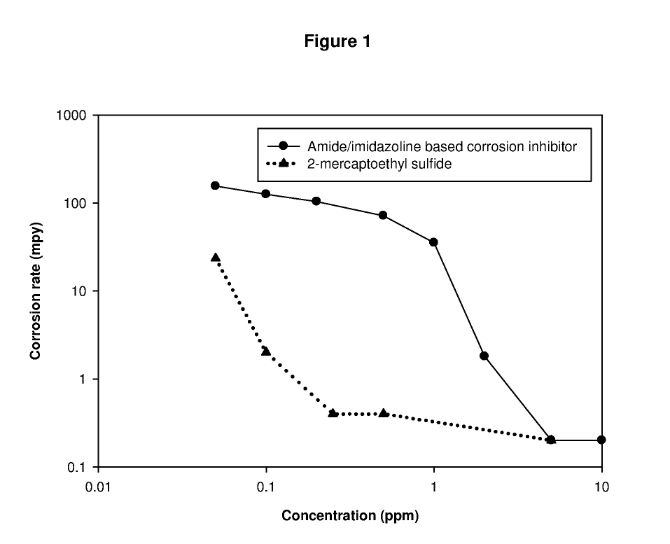

The results of the test are shown in FIG. 1. The data plotted in FIG. 1 is also shown in tabular form in Table 1, comparing concentration of the inhibitor compound (“actives”) on a ppm basis with the level of corrosion measured post-test for each of the inhibitors.

TABLE 1

| | Concentration of actives (ppm) | | | | | | | | |
|---|---|---|---|---|---|---|---|---|---|
| | 0.050 | 0.100 | 0.2 | 0.25 | 0.5 | 1 | 2 | 5 | 10 |
| Amide/imidazoline- | 154.8* | 125.3* | 103.4* | — | 71.3* | 35.2* | 1.8* | 0.2* | 0.2* |
| based inhibitor* | | | | | | | | | |
| (Corrosion rate, mpy) | | | | | | | | | |
| 2-mercaptoethyl sulfide | 23.4 | 2.0 | — | 0.4 | 0.4 | — | — | 0.2 | — |
| (Corrosion rate, mpy) | | | | | | | | | |

*indicates comparative only; not an example of the invention.
— indicates no data available.

What is claimed is:

1. A method of inhibiting corrosion of metals, comprising contacting a metal in an environment such that the metal is corrodible therein and a corrosion inhibitor composition comprising an effective amount of at least one compound selected from the group consisting of one: 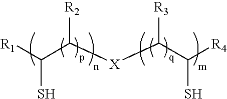
wherein x is carbon, oxygen, nitrogen, or sulfur; R₁, R₂, R₃, and R₄ are independently hydrogen or methyl, m and n are independently integers from 1 to 5, and p and q are independently integers from 1 to 4; 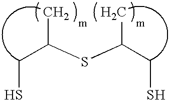
wherein m is an integer from 3 to 4; and 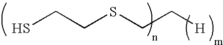
wherein m is an integer from 1 to 4 and n=4−n.

2. The method of claim 1 wherein the metal is iron or steel.

3. The method of claim 1 wherein the environment is a brine, a water-containing hydrocarbon, a water-containing gas, or a combination thereof.

4. The method of claim 1 wherein the compound adheres to the general formula: 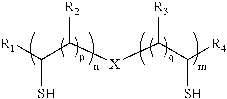
wherein x is carbon, oxygen, nitrogen, or sulfur; R₁, R₂, R₃, and R₄ are independently hydrogen or methyl; and m and n are independently integers from 1 to 4.

5. The method of claim 4 wherein x is sulfur; R₁, R₂, R₃ and R₄ are each hydrogen; and m and n are each the integer 1.

6. The method of claim 1 wherein the effective amount is from about 1 part per billion (ppb) to about 1 percent by volume.

7. The method of claim 6 wherein the effective amount is from about 100 ppb to about 10,000 parts per million (ppm).

8. A method of inhibiting corrosion of metals, comprising contacting a metal in a water-containing hydrocarbon or gas stream, wherein the metal is corrodible, and an effective amount of a corrosion inhibitor composition comprising at least one compound selected from the group consisting of compounds adhering to one of the general formulas: 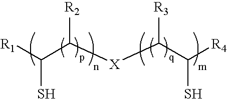
wherein x is carbon, oxygen, nitrogen, or sulfur; R₁, R₂, R₃, and R₄ are independently hydrogen or methyl, m and n are independently integers from 1 to 5, and p and q are independently integers from 1 to 4; 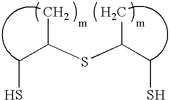
wherein m is an integer from 3 to 4; and 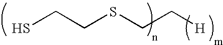
wherein m is an integer from 1 to 4; and n=4−n.

9. The method of claim 8 wherein the metal is iron or steel.

10. The method of claim 8 wherein the water-containing hydrocarbon or gas stream further contains brine.

11. The method of claim 8 wherein the compound adheres to the general formula: 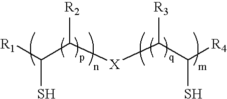
wherein x is carbon, oxygen, nitrogen, or sulfur, R₁, R₂, R₃, and R₄ are independently hydrogen or methyl, m and n are independently integers from 1 to 5, and p and q are independently integers from 1 to 4.

12. The method of claim 11 wherein x is sulfur; R₁, R₂, R₃ and R₄ are each hydrogen; and m and n are each the integer 1.

13. The method of claim 8 wherein the effective amount is from about 1 part per billion (ppb) to about 1 percent by weight.

14. The method of claim 13 wherein the effective amount is from about 100 parts per billion (ppb) to about 10,000 parts per million (ppm).

15. A composition for inhibiting corrosion of a metal in an environment in which the metal is corrodible comprising at least one compound selected from the group consisting of compounds adhering to one of the general formulas: 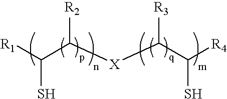
wherein x is carbon, oxygen, nitrogen, or sulfur; R₁, R₂, R₃, and R₄ are independently hydrogen or methyl, m and n are independently integers from 1 to 5, and p and q are independently integers from 1 to 4; 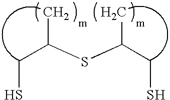
wherein m is an integer from 3 to 4; and 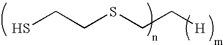
wherein m is an integer from 1 to 4, and n=4−n.

16. The composition of claim 15 wherein x is sulfur; R₁, R₂, R₃ and R₄ are each hydrogen; and m and n are each the integer 1.

17. The composition of claim 15 wherein the metal is iron or steel.
